(19) United States
(12) Patent Application Publication
(10) Pub. No.: US 2025/0116649 A1
(43) Pub. Date: Apr. 10, 2025

(54) DEVICE FOR MONITORING WATER-MISCIBLE COOLING LUBRICANT

(71) Applicant: Matthias LEIPOLDT, Treuen (DE)

(72) Inventor: Matthias LEIPOLDT, Treuen (DE)

(21) Appl. No.: 18/865,639

(22) PCT Filed: May 2, 2023
(86) PCT No.: PCT/EP2023/061455

§ 371 Date: Nov. 13, 2024

(30) Foreign Application Priority Data
May 14, 2022 (DE) 10 2022 001 804.7

(51) Int. Cl. G01N 33/28; G01N 21/41; G01N 21/84; G01N 21/85

(52) U.S. Cl. CPC G01N 33/2888; G01N 21/4133; G01N 21/85; G01N 33/2847; G01N 33/2894; G01N 2021/8416

(57) ABSTRACT

The invention relates to a device for monitoring water-miscible cooling lubricant comprising a chamber (1) and connections (2-5) for water, concentrate and water-miscible cooling lubricant. The connections are connected to the interior of the chamber by means of a mixer (6). The device comprises a refractometer (7), a source of electromagnetic radiation (8) and a digital camera (9) or a radiation detector. The digital camera or the radiation detector is connected to a data processing system (18) that controls the supply of water, the supply of water-miscible concentrate, the supply of water-miscible cooling lubricant of at least one machine tool and the medium-conveying apparatus in such a manner that a particular value or a particular range of the refraction caused by the water-miscible cooling lubricant is achieved and/or maintained and the determined amounts of water, concentrate and water-miscible cooling lubricant are recorded.

The invention relates to a device for monitoring water-miscible cooling lubricants.

Refractometers for determining the refractive index are well known. This involves using the behavior of light at the transition between a prism with known properties and the material to be tested. A prism is used as a measuring prism, which has a known refractive index. The light is scattered at different angles at the transition between the prism and the water-miscible cooling lubricant, allowing the refractive index of the water-miscible cooling lubricant to be measured.

Publication DE 10 2020 028 319 A1 discloses a method and an apparatus for controlling the concentration of water-lubricated cooling lubricants. For this purpose, a refractometer for determining the refractive index of the water-mixed cooling lubricant and a measuring means for measuring the electrical conductivity are arranged in the cooling lubricant circuit of the machine tool. The refractometer is designed as a digital refractometer that has an LED as a light source and a CCD sensor as a detector.

Publication DE 10 2019 206 613 A1 describes an inline refractometer, in particular for ascertaining the water content of a cooling lubricant. The inline refractometer has a prism, an eyepiece and a scale. To determine the water content, the inline refractometer is assigned to a crosspiece of a pipeline. The inline refractometer also has a camera for reading the measured value displayed on the scale. Quantitative detection is not intended.

Publication U.S. Pat. No. 3,838,646 A relates to a noise suppression system for use in a railway marshaling yard. It also relates to a mixing device for producing an oil-in-water emulsion. An unspecified refractometer is used to analyze the solution in the mixing tank.

Publication EP 3 964 834 A1 discloses a method and an apparatus for monitoring a cooling lubricant emulsion. A refractometer with a measuring window is also used for this purpose. An inlet and an outlet of the cooling lubricant emulsion are arranged at an angle to the measuring window.

The invention specified in claim 1 is based on the object of simply automatically monitoring water-miscible cooling lubricant.

This object is achieved by means of the features listed in claim 1.

The device for monitoring water-miscible cooling lubricants is characterized in particular by automatic monitoring of the water-miscible cooling lubricant.

For this purpose, a chamber has a first connection for the particular supply of water, a second connection for the particular supply of water-miscible concentrate, a third connection for the supply of water-miscible cooling lubricant to the machine tool and a fourth connection for discharging the water-miscible cooling lubricant from the chamber. The first connection, the second connection and the third connection are connected to the interior of the chamber by means of a mixer. The interior of the chamber is connected to at least one medium-conveying apparatus. A refractometer is located in the chamber and thus in the water-miscible cooling lubricant. The refractometer is arranged between a source of electromagnetic radiation and a digital camera or radiation detector. The digital camera or the radiation detector is connected to a data processing system that controls the supply of water, the supply of water-miscible concentrate, the supply of water-miscible cooling lubricant of at least one machine tool and the medium-conveying apparatus in such a manner that a particular value or a particular range of the refraction caused by the water-miscible cooling lubricant is achieved and/or maintained and the determined amounts of water, concentrate and water-miscible cooling lubricant are recorded.

Water-miscible cooling lubricants are known to be used for heat dissipation and lubrication during cutting and forming on machine tools. The main components are water and oils. They also contain water-miscible concentrates in the form of emulsifiers and mineral oil as well as additives for better lubrication. Furthermore, mineral oil-free water-miscible cooling lubricants are known, which can contain, for example, canola oil. The oil content of the emulsions is usually between 5 and 8 percent. Water-miscible cooling lubricants must be regularly monitored by the user. The use of water-miscible cooling lubricants improves the service life of the tools and the surface quality of the materials. Therefore, the composition is important in particular when using the water-miscible cooling lubricant. In order to achieve long periods of use, the composition of the water-miscible cooling lubricant must be kept as constant as possible. Used components must be replenished according to use. For this purpose, the water-miscible cooling lubricant must be tested to determine the amount of water and/or concentrate components that need to be added. By checking and adding particular components, the use of the water-miscible cooling lubricant can be extended. This reduces in particular the otherwise high cost of disposal. At the same time, the longer use of the water-miscible cooling lubricant protects the environment. With the device for monitoring water-miscible cooling lubricants, the components to be added can be determined easily and automatically.

For this purpose, the refractometer is used, which has at least one prism. As is well known, the refractometer can be designed according to the principles of transmitted light, grazing incidence or total reflection. A prism with a known refractive index is used. The electromagnetic radiation in the form of light spreads at different angles at the transition between the prism and the water-miscible cooling lubricant. The unknown refractive index of the water-miscible cooling lubricant is measured via the deflection of the electromagnetic radiation. Advantageously, water-miscible cooling lubricant is applied directly to the prism in the chamber. This allows the refractive index of the water-miscible cooling lubricant to be determined directly, the behavior of the electromagnetic radiation at the transition between the prism with known properties and the water-miscible cooling lubricant being used. Advantageously, water-miscible cooling lubricant can flow past so that by adding water and/or concentrate the composition can be changed so that an optimal water-miscible cooling lubricant is again available. The refractive index is characterized by an edge and/or a color change in the image, which is directly converted by means of the digital camera into digital data for processing in the data processing system. The value of the refractive index can thus be determined in relation to the image and/or a measuring scale. For calibration purposes, the lower limit can be assigned to water and the upper limit to concentrate. The measuring scale is located in the beam path of the electromagnetic radiation between the prism and the digital camera. The image can also be converted to digital data via the radiation detector. This can be a one-dimensional line sensor or a two-dimensional array sensor. For example, a CCD sensor can be used as a sensor, where CCD stands for charge-coupled device.

Advantageous embodiments of the invention are set out in the following developments and embodiments. These can further develop the device for monitoring water-miscible cooling lubricants individually or in combination.

In one embodiment, the medium-conveying apparatus can be a pump connected to the fourth connection for conveying water-miscible cooling lubricant from the interior of the chamber. Furthermore, a first valve is connected to the first connection, a second valve to the second connection and a third valve to the third connection. The actuating devices of the first valve, the second valve and the third valve are connected to the data processing system. This ensures a particular supply of water, concentrate and/or water-miscible cooling lubricant. Advantageously, a vacuum pump can be used for this purpose.

In a further development, a first apparatus conveying water as a medium can be connected to the first connection, a second apparatus conveying concentrate as a medium can be connected to the second connection and a third apparatus conveying water-miscible cooling lubricant as a medium can be connected to the third connection. These apparatuses are connected to the data processing system, which controls the conveying apparatuses in such a manner that a particular value or a particular range of the refraction caused by the water-miscible cooling lubricant is achieved and/or maintained.

In a further development, an apparatus measuring the water flow, an apparatus measuring the concentrate flow and an apparatus measuring the cooling lubricant flow can be connected to the data processing system, which controls the water conveying apparatus, the concentrate conveying apparatus and the water-miscible cooling lubricant conveying apparatus in such a manner that a particular value or a particular range of the refraction caused by the water-miscible cooling lubricant is achieved and/or maintained.

Advantageously, water-miscible cooling lubricant of the chamber is located between the source of electromagnetic radiation and a prism of the refractometer.

In one embodiment, the refractometer is advantageously located between the connections arranged in a row at a distance from one another for the supply of water, for the supply of concentrate and for the supply of water-miscible cooling lubricant and the fourth connection for discharging water-miscible cooling lubricant. This allows water-miscible cooling lubricant to flow past the prism of the refractometer.

In one embodiment, the mixer is a tube having at least one spiral arranged in the tube. Said spiral is arranged in a helical shape. Advantageously, two oppositely arranged spirals can also be arranged in the tube.

In one embodiment, an ultrasonic oscillator is located on the chamber in such a manner that ultrasound is introduced into the water-miscible cooling lubricant in the interior of the chamber. This advantageously leads to any deposits that may be present being discharged from the surface of the prism. For this purpose, the ultrasonic oscillator can also be arranged in particular parallel to the prism of the refractometer.

In one embodiment, the refractometer can comprise a cleaning apparatus. This can be a wiper, for example.

By cleaning the refractometer, the device for monitoring water-miscible cooling lubricants can be operated continuously, except for periodic cleaning of the refractometer.

In one embodiment, the device for monitoring water-miscible cooling lubricants can comprise a lux meter for measuring the illuminance of the source of electromagnetic radiation, which lux meter is connected to the data processing system. In conjunction with the image, the function of the device can thus be checked.

In one embodiment, the refractometer is located between the source of electromagnetic radiation and a measuring scale arranged in front of the digital camera. The data processing system is designed in such a manner that the image of the measuring scale and the refractive index from the refraction of the electromagnetic radiation are displayed with a data display means.

In one embodiment, the data processing system can be designed such that the determined amounts of water and concentrate to be supplied to a tank with water-miscible cooling lubricant connected to a machine tool are determined from the determined amounts of water, concentrate and water-miscible cooling lubricant.

An embodiment of the invention is shown in principle in each of the drawings and is described in more detail below.

In the drawings:

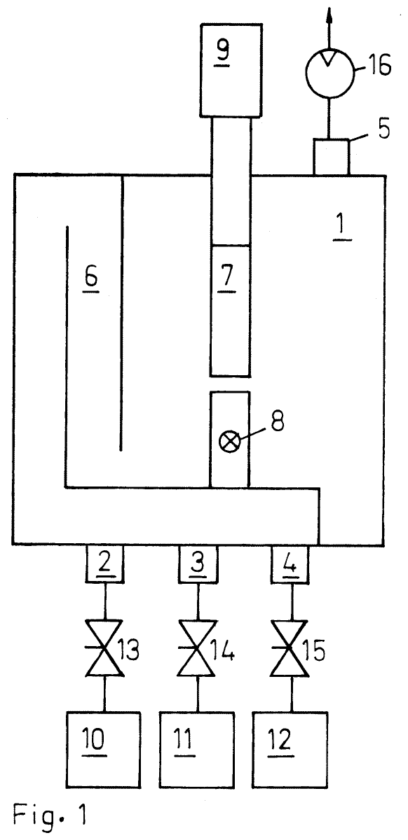

FIG. 1 shows a device for monitoring water-miscible cooling lubricants,

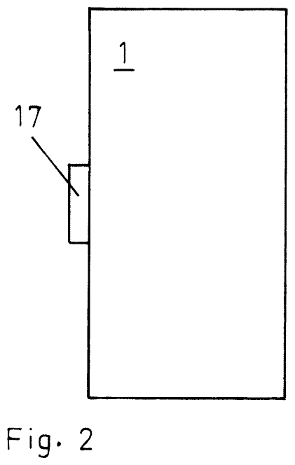

FIG. 2 shows a chamber with an ultrasonic oscillator and

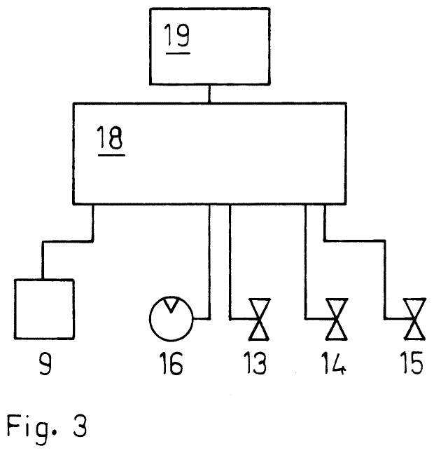

FIG. 3 is a block diagram of the device for monitoring water-miscible cooling lubricants.

A device for monitoring water-miscible cooling lubricants substantially consists of a chamber 1 having a first connection 2, a second connection 3, a third connection 4, a fourth connection 5, a mixer 6, a refractometer 7, a source of electromagnetic radiation 8 and a digital camera 9.

FIG. 1 shows a device for monitoring water-miscible cooling lubricant in a basic illustration.

The chamber 1 has the first connection 2, the second connection 3 and the third connection 4,
the first connection 2 for the particular supply of water 10 being connected to a reservoir with water 10 by means of a first valve 13,
the second connection 3 for the particular supply of water-miscible concentrate 11 being connected to a reservoir with concentrate 11 by means of a second valve 14 and
the third connection 4 for the supply of water-miscible cooling lubricant 4 being connected to a reservoir with water-miscible cooling lubricant 12 of at least one machine tool.

The fourth connection 5 for discharging the water-miscible cooling lubricant 12 from the chamber 1 is connected to a medium-conveying apparatus 16, for example in the form of a vacuum pump 16.

The first connection 2, the second connection 3 and the third connection 4 are connected to the interior of the chamber 1 by means of the mixer 6. The mixer 6 is a tube 6 having at least one spiral arranged in the tube 6, which spiral is helically shaped. Said spiral can be single-pass or intertwined multi-pass. Furthermore, the tube 6 as a mixer 6 can also be arranged in a meandering fashion.

The refractometer 7 is located in the chamber 1 and thus in the water-miscible cooling lubricant 12, the refractometer being arranged between the source of electromagnetic radiation 8 and the digital camera 9 in such a manner that the image from the refraction of the electromagnetic radiation is digitized by means of the digital camera 9. For this purpose, water-miscible cooling lubricant 12 of the chamber 1 is located between the source of electromagnetic radiation 8 and a prism of the refractometer 7. The first connection 2, the second connection 3 and the third connection 4 are arranged in a row, so that the refractometer 7 is located between the connections 2, 3, 4 arranged in a row at a distance from one another for the supply of water 10, for the supply of concentrate 11 and for the supply of water-miscible cooling lubricant 12 and the fourth connection 5 for discharging water-miscible cooling lubricant 12.

FIG. 2 shows a chamber 1 with an ultrasonic oscillator 17 in a basic illustration.

In one embodiment, at least one ultrasonic oscillator 17 can be attached to the chamber 1 in such a manner that the ultrasound is introduced into the water-miscible cooling lubricant 12 in the interior of the chamber 1. For this purpose, the ultrasonic oscillator 17 can be arranged in particular parallel to the prism of the refractometer 7.

FIG. 3 shows a block diagram of the device for monitoring water-miscible cooling lubricant 12 in a basic illustration.

The actuating devices of the first valve 13, the second valve 14, the third valve 15, the apparatus 16 conveying water-miscible cooling lubricant 12 as a medium and the digital camera 9 are connected to a data processing system 18 that controls the supply of water 10, the supply of water-miscible concentrate 11, the supply of water-miscible cooling lubricant 12 of the machine tool and the medium-conveying apparatus 16 in such a manner that a particular value or a particular range of the refraction caused by the water-miscible cooling lubricant 12 is achieved and/or maintained and the determined amounts of water 10, concentrate 11 and water-miscible cooling lubricant 12 are recorded. For this purpose, the data processing system 18 can be connected to a screen 19.

In one embodiment, a lux meter for measuring the illuminance of the source of electromagnetic radiation 8 can be present, which lux meter is connected to the data processing system 18.

In addition, the refractometer 7 can be located between the source of electromagnetic radiation 8 and a measuring scale arranged in front of the digital camera 9. The data processing system 18 can be designed so that the image of the measuring scale and the refractive index from the refraction of the electromagnetic radiation is displayed on the screen 19 as a data display means.

Furthermore, the data processing system 18 can be designed such that the determined amounts of water 10 and concentrate 11 to be supplied to a tank with water-miscible cooling lubricant 12 connected to a machine tool are determined from the determined amounts of water 10, concentrate 11 and water-miscible cooling lubricant 12.

### REFERENCE SIGNS

1 chamber
2 first connection
3 second connection
4 third connection
5 fourth connection
6 tube/mixer
7 refractometer
8 electromagnetic radiation
9 digital camera
10 reservoir with water
11 water-miscible concentrate
12 water-miscible cooling lubricant
13 first valve
14 second valve
15 third valve
16 medium-conveying apparatus
17 ultrasonic oscillator
18 data processing system
19 data display means, e.g., screen

1. A device for monitoring water-miscible cooling lubricant (12), characterized in that a chamber (1) has a first connection (2) for the particular supply of water (10), a second connection (3) for the particular supply of water-miscible concentrate (11), a third connection (4) for the supply of water-miscible cooling lubricant (12) to at least one machine tool and a fourth connection (5) for discharging the water-miscible cooling lubricant (12) from the chamber (1), in that the first connection (2), the second connection (3) and the third connection (4) are connected to the interior of the chamber (1) by means of a mixer (6), in that the interior of the chamber (1) is connected to at least one medium-conveying apparatus (16), in that a refractometer (7) is located in the chamber (1) and thus in the water-miscible cooling lubricant (12), in that the refractometer (7) is arranged between a source of electromagnetic radiation (8) and a digital camera (9) or a radiation detector, and in that the digital camera (9) or the radiation detector is connected to a data processing system (18) that controls the supply of water (10), the supply of water-miscible concentrate (11), the supply of water-miscible cooling lubricant (12) of the machine tool and the medium-conveying apparatus (16) in such a manner that a particular value or a particular range of the refraction caused by the water-miscible cooling lubricant (12) is achieved and/or maintained and the determined amounts of water (10), concentrate (11) and water-miscible cooling lubricant (12) are recorded.

2. The device according to claim 1, characterized in that the medium-conveying apparatus (16) is a pump (16) connected to the fourth connection (5) for conveying water-miscible cooling lubricant (12) from the interior of the chamber (1), in that a first valve (13) is connected to the first connection (2), a second valve (14) to the second connection (3) and a third valve (15) to the third connection (4) and in that the actuating devices of the first valve (13), the second valve (14) and the third valve (15) are connected to the data processing system (18).

3. The device according to claim 1, characterized in that a first apparatus conveying water (10) as a medium is connected to the first connection (2), a second apparatus conveying concentrate (11) as a medium is connected to the second connection (3) and a third apparatus conveying water-miscible cooling lubricant (12) as a medium is connected to the third connection (4) and in that the apparatuses conveying the media are connected to the data processing system (18) that controls the conveying devices in such a manner that a particular value or a particular range of the refraction caused by the water-miscible cooling lubricant (12) is achieved and/or maintained.

4. The device according to claim 3, characterized in that an apparatus measuring the water flow, an apparatus measuring the concentrate flow and an apparatus measuring the cooling lubricant flow are connected to the data processing system (18), which controls the water (10) conveying apparatus, the concentrate (11) conveying apparatus and the water-miscible cooling lubricant (12) conveying apparatus in such a manner that a particular value or a particular range of the refraction caused by the water-miscible cooling lubricant (12) is achieved and/or maintained.

5. The device according to claim 1, characterized in that water-miscible cooling lubricant (12) of the chamber is located between the source of electromagnetic radiation (8) and a prism of the refractometer (7).

6. The device according to claim 1, characterized in that the refractometer (7) is located between the connections (2, 3, 4) arranged in a row at a distance from one another for the supply of water (10), for the supply of concentrate (11) and for the supply of water-miscible cooling lubricant (12) and the fourth connection (5) for discharging water-miscible cooling lubricant (12).

7. The device according to claim 1, characterized in that the mixer (6) is a tube having at least one spiral arranged in the tube.

8. The device according to claim 1, characterized in that at least one ultrasonic oscillator (17) is attached to the chamber (1) in such a way that the ultrasound is introduced into the water-miscible cooling lubricant (12) in the interior of the chamber (1).

9. The device according to claim 1, characterized in that the refractometer (7) has a cleaning apparatus.

10. The device according to claim 1, characterized in that a lux meter is provided for measuring the illuminance of the source of electromagnetic radiation (8) and in that the lux meter is connected to the data processing system (18).

11. The device according to claim 1, characterized in that the refractometer (7) is located between the source of electromagnetic radiation (8) and a measuring scale arranged in front of the digital camera (9) and in that the data processing system (18) is designed in such a manner that the image of the measuring scale and the refractive index from the refraction of the electromagnetic radiation are displayed with a data display means (19).

12. The device according to claim 1, characterized in that the data processing system (18) is designed such that the determined amounts of water (10) and concentrate (11) to be supplied to a tank with water-miscible cooling lubricant (12) connected to the machine tool are determined from the determined amounts of water (10), concentrate (11) and water-miscible cooling lubricant (12).

13. The device according to claim 2, characterized in that a first apparatus conveying water (10) as a medium is connected to the first connection (2), a second apparatus conveying concentrate (11) as a medium is connected to the second connection (3) and a third apparatus conveying water-miscible cooling lubricant (12) as a medium is connected to the third connection (4) and in that the apparatuses conveying the media are connected to the data processing system (18) that controls the conveying devices in such a manner that a particular value or a particular range of the refraction caused by the water-miscible cooling lubricant (12) is achieved and/or maintained.

14. The device according to claim 13, characterized in that an apparatus measuring the water flow, an apparatus measuring the concentrate flow and an apparatus measuring the cooling lubricant flow are connected to the data processing system (18), which controls the water (10) conveying apparatus, the concentrate (11) conveying apparatus and the water-miscible cooling lubricant (12) conveying apparatus in such a manner that a particular value or a particular range of the refraction caused by the water-miscible cooling lubricant (12) is achieved and/or maintained.

15. The device according to claim 14, characterized in that water-miscible cooling lubricant (12) of the chamber is located between the source of electromagnetic radiation (8) and a prism of the refractometer (7).

16. The device according to claim 15, characterized in that the refractometer (7) is located between the connections (2, 3, 4) arranged in a row at a distance from one another for the supply of water (10), for the supply of concentrate (11) and for the supply of water-miscible cooling lubricant (12) and the fourth connection (5) for discharging water-miscible cooling lubricant (12).

17. The device according to claim 16, characterized in that the mixer (6) is a tube having at least one spiral arranged in the tube.

18. The device according to claim 17, characterized in that at least one ultrasonic oscillator (17) is attached to the chamber (1) in such a way that the ultrasound is introduced into the water-miscible cooling lubricant (12) in the interior of the chamber (1).

19. The device according to claim 18, characterized in that the refractometer (7) has a cleaning apparatus.

20. The device according to claim 19, characterized in that:
a lux meter is provided for measuring the illuminance of the source of electromagnetic radiation (8) and in that the lux meter is connected to the data processing system (18);
the refractometer (7) is located between the source of electromagnetic radiation (8) and a measuring scale arranged in front of the digital camera (9) and in that the data processing system (18) is designed in such a manner that the image of the measuring scale and the refractive index from the refraction of the electromagnetic radiation are displayed with a data display means (19); and
the data processing system (18) is designed such that the determined amounts of water (10) and concentrate (11) to be supplied to a tank with water-miscible cooling lubricant (12) connected to the machine tool are determined from the determined amounts of water (10), concentrate (11) and water-miscible cooling lubricant (12).
